(19) United States
(12) Patent Application Publication
(10) Pub. No.: US 2005/0003025 A1
(43) Pub. Date: Jan. 6, 2005

(54) Method of mitigating the odor of valerian extracts

(76) Inventors: Robert D. Hargens, Lincoln, NE (US); Yimin Jia, Lincoln, NE (US); Steve E. Jurgens, Filley, NE (US)

Correspondence Address: NOVARTIS;CORPORATE INTELLECTUAL PROPERTY, ONE HEALTH PLAZA 430/2, EAST HANOVER, NJ, 07936-1080, US

(21) Appl. No.: 10/880,397

(22) Filed: Jun. 29, 2004

Related U.S. Application Data
(60) Provisional application No. 60/484,072, filed on Jun. 30, 2003.

(51) Int. Cl. A61K 35/78

(52) U.S. Cl. 424/733

(57) ABSTRACT

A method for mitigating the malodor normally associated with a valerian extract. The method consists of adjusting the apparent pH of an aqueous-alcoholic solution of the valerian extract with a chemical base in order to neutralize the valerian extract. The extract can then be subsequently mixed with a carrier to form a powder. Alternatively, the deodorized valerian extract can be used in pharmaceutical, nutritional or dietary formulations.

### FIELD OF THE INVENTION

The present invention relates to a method of mitigating the odor of Valerian (Valeriana officinalis L.) extracts. The present invention also relates to a deodorized valerian resulting from such a method that is suitable for use in pharmaceutical and/or nutritional compositions.

### BACKGROUND OF THE INVENTION

Valeriana officinalis L., a plant more commonly known as valerian, has been recognized for centuries as having significant medicinal properties when taken orally or used topically. Many have isolated extracts of the plant and used such extracts in oral dietary supplements in the form of tablets or soft capsules. When taken orally, these extracts have been used as sedatives, relaxants and spasmolytics. When applied topically, these extracts have been used as washes for sores and pimples.

Extracts of valerian contain many constituents. Although not all of the constituents have been isolated and characterized, of the known constituents a significant number of them are biologically active. To date, the known chemical constituents include, but are not limited to, bicyclic monoterpenes and their derivatives (e.g., valerenic acid, valerianal, valerianone, hydroxyvalerenic acid, acetoxyvalerenic acid); valepotriates (e.g., valtrate, isovaltrate, acevaltrate and valerosidatum); alkaloids (e.g., valerianine, actinidine); baldrinal; bronyl acetate; bronyl isovalerianate; flavonoids (i.e., polyphenols); tannins (e.g., tannic acid, gallotannic acid); and triterpenes. Although, the specific role that each of these constituent compounds plays in the biological activity of valerian is to date unknown, some attribute the biological activity to the valerenic acid and/or valepotriates.

Despite valerian's medicinal properties, use of valerian has been limited because of its malodor. Extracts of valerian exhibit a strongly disagreeable and unpleasant malodor that has been likened to “dirty socks” or “rotten cheese.” As with the biological activity, it is not known which constituents of valerian are primarily responsible for the malodor. However, some believe that the malodor arises from acids present in the valerian. See, for example, U.S. Patent Application Ser. No. 60/173,983 filed on Dec. 30, 1999 (hereinafter, the “'983 application”).

In attempting to reduce the odor of valerian, a method of mixing a chemical base to the concentrate of valerian is disclosed in the '983 application. It is believed, according to the '983 application, that the chemical base and the acids of the valerian concentrate form a salt resulting in a concentrate which is less odoriferous than the original extract.

As disclosed in Example 1 of the '983 application. The valerian biomass is added to a solvent that includes a mixture of 70% denatured ethanol (95% ethanol and 5% methanol) and 30% water. This mixture is then concentrated under a reduced pressure at approximately 50° C. until it forms an oily consistency. The mixture begins as 2 L of solvent solution for each 1 kg of chipped valerian biomass and concentrates to a final volume of approximately 0.16 L for each 1 kg of chipped valerian root extracted. One of ordinary skill in the art would recognize that the concentrate prior to mixing with the chemical base contains no alcohol since the alcohol would have evaporated during the concentration process.

The present invention take a different approach than the process disclosed in the '983 application by enhancing the reaction conditions between the base and the acids. These improved conditions result in a concentrate that is less odoriferous than that of the '983 application.

Thus, there is a need to have a method that can further improve on methods of mitigating or minimizing the malodor associated with valerian. The present invention addresses that need.

### SUMMARY OF THE INVENTION

The present invention relates to a method of producing a valerian extract, in dried or liquid form, that has the odor normally associated therewith mitigated. The method includes the step of contacting a base with an aqueous-alcoholic solution or mixture containing valerian constituents. The apparent pH of the aqueous-alcoholic extract is adjusted by the base until the apparent pH is within a range of from about 7 to about 12, e.g., about 8 to about 10. Alternatively, the base is added to the aqueous-alcoholic extract until a sufficient amount (as hereinafter defined) of the acids contained within the valerian extract is converted into salts (the base reacts with the acids to form such salts). The alcohol in the base-treated extract can be subsequently separated or removed, e.g., by distillation removed. The remaining aqueous base-treated extract (i.e., liquid extract) can be mixed with a carrier and subsequently dried to from a dried extract, or powder. The resulting deodorized liquid extract, paste and/or dried extract has less odor than a valerian extract that was not subjected to the base neutralization process of the present invention.

Other features and advantages of the present invention will be apparent from the description of the invention and from the claims.

### DETAILED DESCRIPTION

The present invention relates to a method of mitigating the malodor often associated with valerian extracts. The present invention also includes a composition that includes valerian that has been subjected to the aforementioned process.

Valerian is native to the Americas, Asia and Europe, and belongs to the valerianaceae family and is technically known as Valeriana officinalis L. As used herein, the term “valerian”-encompasses Valeriana officinalis L. as well as its subspecies including, but not limited to, Valeriana exaltata J.C. Mikan, Valeriana nitida Kreyer, Valeriana palustris Wibel, Valeriana wolgenis Kazak, Valeriana grossheimii Vorosch, Valeriana collina Wallr, Valeriana Rossica P.A. Smirn, Valeriana spryngini P.S. Smirn, Valeriana angustifolia Tuasch, Valeriana tenuifolia Vahl, Valeriana wallrothii Kreyer, Valeriana ucrainica Demjan, Valeriana sambucifolia J.C. Mikan, Valeriana excelsa Poir, and Valeriana officinalis L.supsp. excelsa (Poir.) Rouy. Common names of valerian include valerian, garden valerian, great wild valerian, capon's tail, setwall, garden heliotrope, St. George's herb, amantilla, phu, tobacco root and vandal root.

The “therapeutic benefits” of valerian, as defined herein, include, but are not limited to, its sedative and sleep inducing activity as well as its spasmodic and myorelaxant activity

Valerian appears as tall, wispy plants with dark green leaves that are pointed at the tip and hairy underneath. When in bloom, valerian has white, light purple or pink flowers. It is typically the roots, rhizomes and stolons of the plant that are used for their therapeutic benefits. As used herein, the term “plant biomass” refers to the parts of the valerian used that possesses any of constituents responsible for the therapeutic benefits. Plant biomass, for example, includes the roots which refer to the part of a plant that is normally underground responsible for nutrient absorption and anchoring the plant into the ground. Plant biomass also includes rhizomes which are horizontal, usually underground stems that often send out roots and shoots from its nodes.

As used herein the term “odor” refers to the property of valerian biomass after the valerian has been harvested as known by or familiar to one of ordinary skill in the art (including extracts and dried extracts thereof) that manifests a physiological sensation in the olfactory nervous system of a human without the aid of any electrical, mechanical, or electromechanical equipment or devices.

As used herein the term “mitigation” and “mitigating” refers to the lessening, reduction or elimination of an odor as compared to any prior art compound, extract or form of valerian as perceived by a human. As used herein, the term “deodorized” means mitigated.

As used herein the term “extraction” refers to a process for separating a mixture of chemical constituents (e.g., the constituents responsible for the therapeutic benefits prepared from valerian) from the plant biomass. By using different solvents, constituents with different solubilities and adsorption strengths can be selectively extracted. The term “extract” refers to a solution of the solvent and the constituents of valerian solubilized therein and extracted thereby. The term “paste” refers to a concentrated extract such that the all or a majority of the non-aqueous solvent component has been separated or removed from the remaining aqueous solvent. The term “dried extract” refers to the dried solute of the extract with the solvent removed, for example by evaporation.

The term “pH” as used herein is the acidity and/or alkalinity of a solution quantitatively expressed as the negative logarithm of the hydrogen ion concentration of that solution. The term “apparent pH” refers to the pH of a solution of an aqueous and non-aqueous solution, for example, an aqueous-alcoholic solution of water and ethanol. Because the solution is not a hundred percent water, a measured pH for an aqueous-non-aqueous solution is different than that of water alone. This different pH is termed as the “apparent pH.” For example, in order to define a neutral pH in a solution of 70% ethanol, ethanol is added to a standardized pH 7.0 buffer solution until the final solution is 70% alcohol and 30% buffer. The pH of this solution is then measured. The apparent pH of 70% alcohol is 8.4 which is equivalent to an aqueous pH of 7. Table 1, set forth below, shows the relationship between the pH and apparent pH of water and a 70% ethanol solution (note that all of the number in Table 1 are approximations). The apparent pH can change depending on the non-aqueous component of the solution. For example, the apparent pH of a 70% ethanol solution is not identical to a 70% methanol solution which is not identical to 70% isopropanol solution. However, one of ordinary skill in the art can determine the relationship between water and the non-aqueous/aqueous solution by using the method disclosed above.

TABLE 1

| pH of Water | apparent pH of 70% ethanol |
|---|---|
| 3.0 | 4.0 |
| 3.7 | 4.8 |
| 6.5 | 8.0 |
| 7.0 | 8.4 |
| 7.4 | 9.0 |
| 8.3 | 10.0 |
| 9.2 | 11.0 |

As used herein the term “base” refers to any compound or mixture of compounds that can effectively raise the pH of a neutral solution to above 7.0. The base can react with an acid to form a salt. Any base that neutralizes an acid and that is also suitable for use in oral preparations that are to be ingested by a mammal, especially a human, can be used in the present invention. Examples of bases include, but are not limited to, inorganic bases (e.g., potassium hydroxide, sodium hydroxide, calcium carbonate, potassium carbonate and sodium bicarbonate) and organic bases (e.g., diethanolamine, triethanolamine).

To facilitate the subsequent extraction steps, for example by increasing the surface area of the plant biomass, the particle size of the plant biomass is reduced. Either fresh or dried plant biomass can be used. If dried plant biomass is used, any method of drying known in the art is suitable. For example, the plant biomass can be sun-dried, dried by heated air or by lyophilization until the moisture and/or liquid content of the plant biomass is less than ten percent. After drying, the plant biomass can be reduced into smaller particles by using any known method of crushing, chopping, pounding or comminuting. Equipment useful for reducing the size of the plant biomass includes, but not limited to, commercial grinders, chippers, shredders and hammer mills. Once reduced, the particle sizes of the ground plant biomass can range from about 0.01 mm to about 1 mm.

In general terms, a suitable method of extraction of the constituents responsible for the therapeutic benefits of valerian is the addition of a solvent to the fresh or dried valerian plant biomass. Suitable extraction solvents include, but are not limited to, water, dilute acids, organic solvents, supercritical or near supercritical fluid solvents (e.g., carbon dioxide, nitrous oxide, propane, ammonia, ethane, fluorohydrocarbons) and any mixtures or combinations thereof. Organic solvents, for example, include, but are not limited to, butane, hexane, acetone, methanol, ethanol, propanol, isopropanol, butanol, methylene chloride, dichloromethane, acetonitrile, ethyl acetate, butyl acetate, xylene, toluene or any mixture thereof.

In particular, alcoholic solutions are particularly useful extraction solvents in the present invention. For example, an alcoholic solution comprises from about 30% (v/v) to about 100% (v/v) alcohol and between about 70% (v/v) to about 0% (v/v) water. Examples of such alcoholic solutions include, but are not limited to, about 30% (v/v) alcohol and about 70% (v/v) water; about 40% (v/v) alcohol and about 60% (v/v) water; about 50% (v/v) alcohol and about 50% (v/v) water; about 60% (v/v) alcohol and about 40% (v/v) water; about 70% alcohol and about 30% (v/v) water; about 80% (v/v) alcohol and 20% (v/v) water; and about 90% (v/v) alcohol and 10% (v/v) water. The alcohol used in the alcoholic solutions is fully miscible in water and can be any C₁-C₆ alcohol, for example, methanol, ethanol, n-propanol, and isopropanol, n-butanol, isobutanol.

The mixture of the plant biomass and the extraction solvent (i.e., the extraction solution) can be stirred or agitated by any conventional methods, for example mechanical methods. Examples of mixing means include, but are not limited to, magnetic stirrers, overhead stirrers, mixers, blenders and sonicators. Sufficient agitation time is needed to allow the plant biomass and the solvent to be well mixed, for example for about two hours when using a blender. The agitation temperature should be below 40° C. to minimize any degradation of the constituents in the plant biomass.

After agitation, the extraction solution can then be separated from the residual plant biomass by an appropriate separation procedure such as, for example filtration and/or centrifugation to recover the extract. For example, filtration through a mesh screen with a mesh size of from about 80 to about 200 can be used.

If the extraction solvent were not alcohol, then the extraction solvent is separated from the extraction solution using means known in the art. If the extraction solvent is alcohol, then the extraction solution can be further concentrated by separating the alcohol from the water through, for example a distiller, to form a valerian paste. The concentration step is optional since alcohol is subsequently added back to the valerian paste which will be made clear below.

The valerian liquid extract, paste, or dried extract can contain one or more constituents responsible for the therapeutic benefits of valerian. Of these constituents, it is believed that the acids, are responsible for the malodor associated with valerian. Examples of acids found in valerian include, but are not limited to valeric acid, isovaleric acid, acetoxyvalerenic acid, hydroxyvalerenic acid, and flavonoids.

Valeric acid is also known as pentanoic acid, valerianic acid and propylacetic acid Acetoxyvalerenic acid is also known as acetoxyvalerianic acid Hydroxyvalerenic acid is also known as hydroxyvalerianic acid. Isovaleric acid is also known as 3-methylbutanoic acid, delphinic acid and isovalerianic acid. Flavonoids refer to polyphenols that have a carbon skeleton.

Prior to contacting the valerian paste with a base, the valerian paste is reconstituted, or rediluted, with a new solvent that includes at least one alcohol and, optionally, water, (the “neutralization solvent”). The neutralization solvent, for example, is an aqueous-alcohol solvent. For example, the neutralization solvent comprises from about 10% (v/v) to about 90% (v/v) alcohol and between about 90% (v/v) to about 10% (v/v) water. A further example of the content of the neutralization solvent is from about 50% (v/v) to about 80% (v/v) alcohol and between about 50% (v/v) to about 20% (v/v) water. Yet another example of the neutralization solvent is about 70% (v/v) alcohol and about 30% (v/v) water. The alcohol used in the neutralization solvent is fully miscible in water and can be any C₁-C₆ alcohol, for example, methanol, ethanol, n-propanol, and isopropanol, n-butanol, isobutanol and mixtures or combinations thereof. For example the alcohol can be 190 proof S.D.A./3.A alcohol which comprises for every 105 gallons, 100 gallons of ethanol and 5 gallons of methanol. The volume percentage of ethanol (200° basis) at 60° F. is 90.5. Hereinafter, this alcohol is termed “190 proof SDA alcohol.”

If the extraction solvent previously used contained either methanol or ethanol, then the methanol or ethanol in that extraction solvent would not have to be removed prior to mixing the extract with the neutralization solvent since the neutralization solvent also contains methanol and/or ethanol. Thus, the neutralization solvent already incorporates the extraction solvent.

The valerian paste is mixed with the neutralization solvent using any conventional means of means of mixing or agitation for about thirty minutes to about an hour depending on the batch size that is to be mixed or agitated.

The neutralization solvent containing the valerian constituents is then contacted with a base. A sufficient quantity of base should be used such that the a substantial amount of the acids present in the extracted valerian constituents are converted into salts. As used herein, “substantial amount” refers to at least ninety percent. Measuring the apparent pH of the neutralization solvent after a base has been added can indicate 0.7 when the acids are converted into salts. For example, the initial apparent pH is about 4 to 6 for a re-constituted and well mixed valerian extract in the neutralization solvent (70% alcohol). This initial apparent pH is dependent on the amount of the acids present in the extracted valerian constituents. For example, the targeted final apparent pH of the mixture is from about 7 to 12, for example 8 to 10, or about 9 to about 9.5. The result of contacting with a base is a “base-treated extract.

Without being wished to be bound by theory, it is believed that having the base contact a solution of valerian constituents that contains both water and alcohol optimizes the solubility of all the acids that make up the valerian constituents. This optimization of the solubility permits all of the acids to contact the base and subsequently be converted into salts.

The base-treated extract, which has now been deodorized, can be further concentrated or dried to form a free-flowing powder. To concentrate the base-treated extract, the water and/or alcohol can be separated, for example by evaporating or distilling.

To make a free-flowing powder of the base-treated extract, a carrier is dispersed into the base-treated extract. Examples of carriers, include, but are not limited to, maltodextrin, starch (e.g., sodium starch octenylsuccinate), tricalcium phosphate or silicon dioxide. The carrier should be of a food-grade or suitable for oral ingestion, i.e., non-toxic. To disperse the carrier in the base-treated extract, a colloid mill, homogenizer, blender or similar equipment can be used. The dispersion can then be spray-dried, freeze-dried or vacuum dried to form a free flowing powder.

Compositions suitable for incorporating the deodorized valerian extract include, but are not limited to, pharmaceuticals, nutraceuticals, dietary supplements and cosmetics. Such compositions can be administered to a mammal, especially a human, orally, parenterally, transdermally, transmucosally, intranasally, bucally or rectally. Suitable delivery vehicles for such compositions include, but are not limited to, sachets, soft gels, powders, syrups, pills, capsules, tablets, inhalants, implants, liquid drops, sublinguals, injectables, patches, suppositories, suspensions, emulsions and solutions.

These compositions can be prepared by using methodology that is well known by an artisan of ordinary skill. For instance, see Remington's Pharmaceutical Sciences, 18^{th} Ed. (Mack Publishing Co. 1990) hereby incorporated by reference in its entirety. For example, for an oral pharmaceutical composition, the odor-mitigated valerian extract is combined with edible pharmaceutically acceptable solid or liquid carriers and or excipients such as fillers (e.g., cellulose, lactose, sucrose, mannitol, sorbitol and calcium phosphates); binders (e.g., starch, gelatin, tragacanth, methyl cellulose and polyvinyl pyrrolidone); flow agents (e.g., silicic acid, silicon dioxide, talc, stearic acid, magnesium stearate, calcium stearate); diluents (e.g., polyethylene glycol); disintegrating agents; coloring agents, flavoring agents, dyes and pigments.

The following examples are illustrative, but do not serve to limit the scope of the invention described herein. They are meant only to suggest a method of practicing the present invention.

### EXAMPLE 1

97 gm of valerian plant biomass in the form of a valerian paste obtained from PureWorld Botanicals (South Hackensack, N.J.) is weighed and transferred into a 4000 ml flask. In the same flask, 910 ml of 190 proof SDA alcohol is added. Subsequently, water deionized is added until the 1300 ml mark on the flask is reached. The mixture of the alcoholic solution and valerian plant biomass is stirred magnetic stirrer for about thirty minutes.

Separately, a 1 M solution of potassium carbonate is added incrementally to the flask. A pH meter is used to monitor the apparent pH of the solution. A total of 33 ml of potassium carbonate solution is used to reach an apparent pH of 9. During the pH adjustment, large amounts of bubbles may be observed. The odor of the valerian is lesser than prior to the addition of the potassium carbonate solution; however a residual valerian odor can still be detected. An additional 9 ml of potassium carbonate is added resulting in a final apparent pH of 10.2. After waiting an hour, the pH settles at around 0.7.

The alcohol is subsequently evaporated using a ROTOVAP rotary evaporator at a temperature below 40° C. under reduced vacuum. No valerian odor is detected.

### EXAMPLE 2

97 gm of valerian plant biomass in the form of a valerian paste obtained from PureWorld Botanicals (South Hackensack, N.J.) is weighed and transferred into a 4000 ml flask. In the same flask, 910 ml of 190 proof SDA alcohol is added. Subsequently, water deionized is added until the 1300 ml mark on the flask is reached. The mixture of the alcoholic solution and valerian plant biomass is stirred magnetic stirrer for about thirty minutes.

Separately, a 1 M solution of potassium carbonate is added incrementally to the flask. A pH meter is used to monitor the apparent pH of the solution. A total of 33 ml of potassium carbonate solution is used to reach an apparent pH of 9. The odor of the valerian is lesser than prior to the addition of the potassium carbonate solution; however a residual valerian odor can still be detected. An additional 9 ml of potassium carbonate is added resulting in a final apparent pH of 10.2. After waiting an hour, the pH settles at around 9.7. No valerian odor is detected.

The alcohol is subsequently evaporated using a ROTOVAP rotary evaporator at a temperature below 40° C. under vacuum

### EXAMPLE 3

91 gm of valerian plant biomass in the form of a valerian paste obtained from PureWorld Botanicals (South Hackensack, N.J.) is weighed and transferred into a 4000 ml flask. In the same flask, 1220 ml of a 70% 190 proof SDA alcohol is added. The mixture of the alcoholic solution and valerian plant biomass is stirred magnetic stirrer for about thirty minutes.

Separately, a 1 M solution of potassium carbonate is added incrementally to the flask. A pH meter is used to monitor the apparent pH of the solution. A total of 51 ml of potassium carbonate solution is used to reach an apparent pH of 9. The odor of the valerian is lesser than prior to the addition of the potassium carbonate solution; however a residual valerian odor can still be detected. An additional 9 ml of potassium carbonate is added resulting in a final apparent pH of 10.2. After waiting an hour and a half, the pH settles to about 9.

The alcohol is subsequently evaporated using a ROTOVAP rotary evaporator at a temperature below 40° C. under vacuum.

### EXAMPLE 4

200 kg of valerian plant biomass in the form of a valerian paste obtained from PureWorld Botanicals (South Hackensack, N.J.) is weighed and transferred into a 1,000-gallon vacuum still. A total of 160 gallons (about 606 L) of 190 proof SDA alcohol. After the solution is mixed, a sample of the solution is taken to have its apparent pH measured. The apparent pH is about 5.2.

A total of 115 kg of a 1 N solution of potassium hydroxide is added to the still with continuous agitation. Once again, a sample of the solution is taken to have its apparent pH measured. The apparent pH is about 9.6. The alcohol is then distilled from the solution by using a vacuum (about 711 mm of Hg) at a temperature below 40° C. After distillation, a total of 273 kg of extract is obtained and the pH is about 7.9.

### EXAMPLE 5

200 kg of valerian plant biomass in the form of a valerian paste obtained from PureWorld Botanicals (South Hackensack, N.J.) is weighed and transferred into a 1,000-gallon vacuum still. A total of 160 gallons (about 606 L) of 190 proof SDA alcohol. After the solution is mixed, a sample of the solution is taken to have its apparent pH measured. The apparent pH is about 5.2.

A total of 115 kg of a 1 N solution of potassium hydroxide is added to the still with continuous agitation. Once again, a sample of the solution is taken to have its apparent pH measured. The apparent pH is about 9.6. The alcohol is then distilled from the solution by using a vacuum (about 711 mm of Hg) at a temperature below 40° C. After distillation, a total of 273 kg of extract is obtained and the pH is about 7.9.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

1. A method for mitigating the malodor associated with a valerian extract comprising the step of contacting said extract with a sufficient amount of a base to form a base-treated extract, wherein said valerian extract comprises valerian biomass, water and alcohol.

2. The method of claim 1, wherein said base is an inorganic base selected from the group consisting of potassium hydroxide, sodium hydroxide, calcium carbonate, potassium carbonate and sodium bicarbonate.

3. The method of claim 2, wherein said base is potassium hydroxide.

4. The method of claim 1, wherein said sufficient amount is a quantity able to convert a substantial amount of acid in said valerian extract into salts.

5. The method of claim 1, wherein said alcohol comprises ethanol.

6. The method of claim 5, wherein said base-treated extract has an apparent pH from about 7 to about 12.

7. The method of claim 6, wherein said apparent pH is from about 8 to about 10.

8. The method of claim 1, wherein said alcohol comprises from about 10% to about 90% (v/v) of said valerian extract.

9. The method of claim 8, wherein said alcohol comprises from about 50% to about 80% (v/v) of said valerian extract.

10. A method for producing a deodorized extract of valerian from valerian biomass comprising the steps of:
extracting said valerian biomass with a solvent comprising water and an alcohol resulting in a valerian extract;
contacting said valerian extract with a sufficient amount of a base resulting in a base-treated extract;
separating said alcohol from said base-treated extract resulting in a liquid extract.

11. The method of claim 10, further comprising the step of recovering said odor-mitigated extract.

12. The method of claim 10, wherein said separating is accomplished by distillation.

13. The method of claim 10, wherein said sufficient amount is a quantity able to convert a substantial amount of acid in said valerian extract into salts.

14. The method of claim 13, wherein said base is an inorganic base selected from the group consisting of potassium hydroxide, sodium hydroxide, calcium carbonate, potassium carbonate and sodium bicarbonate.

15. The method of claim 10, wherein said alcohol comprises from about 10% to about 90% (v/v) of said valerian extract.

16. A method for producing a deodorized extract of valerian from valerian biomass comprising the steps of:
extracting said valerian biomass with a solvent comprising water and a first alcohol resulting in a valerian extract;
separating said first alcohol from said valerian extract resulting in a valerian paste;
adding a second alcohol to said valerian paste resulting in an aqueous-alcoholic valerian extract;
contacting said aqueous-alcoholic valerian extract with a sufficient amount of a base resulting in a base-treated extract;
separating said second alcohol from said base-treated extract resulting in a liquid extract.

17. An odor-mitigated extract produced by the process of claim 16.

18. An odor-mitigated extract produced by the process of claim 1.

19. A method for mitigating the malodor associated with a valerian extract comprising the step of contacting said extract with a sufficient amount of a base in the presence of an alcohol.

20. The method of claim 19, wherein said alcohol comprises from about 10% to about 90% (v/v) of said valerian extract.

21. The method of claim 20, wherein said alcohol comprises from about 50% to about 80% (v/v) of said valerian extract.
